(19) United States
(12) Patent Application Publication
(10) Pub. No.: US 2009/0104258 A1
(43) Pub. Date: Apr. 23, 2009

(54) Use of tocopheryl phosphate as an agent for preventing or slowing down the appearance of the effects of skin ageing

(75) Inventors: Marc Dumas, Saint Jean le Blanc (FR); Emmanuelle Noblesse, Donnery (FR); Valerie Alard, Orleans (FR); Dominique Quiles, Chemault (FR); Eric Perrier, Les Cotes D'Arey (FR)

Correspondence Address: HAMRE, SCHUMANN, MUELLER & LARSON, P.C., P.O. BOX 2902, MINNEAPOLIS, MN, 55402-0902, US

(73) Assignee: LVMH RECHERCHE, Saint Jean de Braye (FR)

(21) Appl. No.: 12/284,851

(22) Filed: Sep. 25, 2008

(30) Foreign Application Priority Data
Sep. 26, 2007 (FR) 0706750

(51) Int. Cl. A61K 8/49; A61K 9/127; A61Q 19/08

(52) U.S. Cl. 424/450; 514/100

(57) ABSTRACT

The invention relates to a cosmetic composition of a tocopheryl phosphate, especially in its dl or d form, or in the form of a cosmetically acceptable salt or ester thereof, as a cosmetic agent for preventing or slowing down the appearance of the effects of skin ageing, in particular of photoageing of the skin, said tocopheryl phosphate being at least partially encapsulated in lipid vesicles, in particular liposomes. It relates most particularly to the use of alpha-tocopheryl phosphate. It also relates to cosmetic compositions containing a tocopheryl phosphate, in particular an alpha-tocopheryl phosphate, at least partially incorporated into lipid vesicles, in particular liposomes, these compositions being in particular in the form of a serum or of a stable emulsion. The invention also relates to a cosmetic skincare process.

The invention relates to a novel use of tocopheryl phosphate, in particular in its dl or d form, or of an ester thereof or of a salt thereof, which is cosmetically or pharmaceutically acceptable, in the cosmetics field, and also to the compositions containing same and to the cosmetic methods using them.

Each day, the skin is subjected to considerable mechanical stresses.

Observations have shown that skin ageing leads to a loss of cell density in the superficial layers of the skin.

The loss of tissue material (collagen and other dermal matrix proteins) brought about by this absence of cells is reflected by less tissue cohesion and also by a thinner epidermis with fewer cell strata.

The stress to which the skin is subjected, in particular UV exposure, leads to a deterioration and then a natural elimination of numerous epidermal and dermal cells.

The many histological observations made in order to understand the key phenomena of dermal ageing have in particular made it possible to conclude that there is a loss of dermal cell density in the wrinkled light-exposed areas.

This loss largely explains the loss of tissue material (collagens and other proteins of the dermal matrix and of the dermal-epidermal junction) that is observed during ageing, since this material can no longer be produced and deposited under the wrinkle by absent cells.

At the epidermal layer, under the wrinkles, this depletion of cells is also observed (Contet-Audonneau et al., A histological study of human wrinkle structures: comparison between sun-exposed areas of the face, with or without wrinkles, and sun-protected areas. Br J Dermatol. 1999 June; 140(6):1038-47) and is reflected, at the histological level, by a thinner epidermis with fewer cell strata in the hollow of the wrinkles.

This phenomenon involves most particularly the cells of the epidermal basal layer.

In fact, although the considerable undulations of the dermal-epidermal junction (DEJ) in young skin support a large basal cell population, these undulations of the DEJ disappear with age, this interface then supporting only a small basal cell population.

Furthermore, preliminary observations imply that, in wrinkled light-exposed areas, a similar phenomenon of decrease in the basal cell population occurs.

This phenomenon, which affects in particular the cells of the basal layers of the epidermis, is important because it is these cells which ensure the formation and the renewal of the epidermal cells, and are themselves derived from the division of “epidermal stem cells”.

These epidermal stem cells are present in the basal layers of the epidermis and are defined as being cells with a high regenerative potential, which have the particularity of being able to give an unlimited descendance of “daughter” cells.

Maintaining these epidermal stem cells in the basal layers of the epidermis is therefore a major target for preventing or slowing down the loss of cell density in the epidermis as observed during photoageing of the skin.

The Applicant has now shown that it is possible to protect the stem cells of the epidermis by means of a treatment using tocopheryl phosphate, and in particular alpha-tocopheryl phosphate, especially in its dl or d form, or an ester thereof or a salt thereof, which is cosmetically or pharmaceutically acceptable.

Tocopheryl phosphate is a molecule which was described by the Applicant, in patent application WO 91/11189, for its use in the preparation of a pharmaceutical or cosmetic or dermatological composition for the prevention or treatment of allergic manifestations such as skin allergy or bronchial or inflammatory asthma, or else for the prevention or treatment of the harmful effects of free radicals.

By virtue of the studies by the inventors of the present application, it has been shown that tocopheryl phosphate, in particular alpha-tocopheryl phosphate, can be used as an active agent in cosmetic compositions which aim at preserving the basal cells of the epidermis and the epidermal stem cells thereof, which are the only cells capable of dividing and of giving numerous generations of “daughter” cells, and thus of having a positive impact on epidermal regeneration, reflected by the maintaining of a sufficient cell density in the various layers of the epidermis and of the protein constituents that these cells produce.

The present invention thus relates to the use, in cosmetic compositions, of tocopheryl phosphate, and in particular of alpha-tocopheryl phosphate, especially in its dl or d form, or of an ester thereof or of a salt thereof, which is cosmetically or pharmaceutically acceptable, as an active agent for preventing or slowing down skin ageing related in particular to the effects of UV exposure.

According to a second aspect, the invention also relates to new cosmetic compositions containing a tocopheryl phosphate as defined above.

According to a third aspect, it also relates to a cosmetic skincare process for preventing or slowing down the appearance of the effects of skin ageing by applying, to the parts of the latter that are involved, a composition containing, as active agent, a tocopheryl phosphate, in particular an alpha-tocopheryl phosphate.

More specifically, according to an essential characteristic of its first aspect, the invention relates to the use, in a cosmetic composition, of a tocopheryl phosphate, especially in its dl or d form, or in the form of a cosmetically acceptable salt or ester thereof, as a cosmetic agent for preventing or slowing down the appearance of the effects of skin ageing, in particular photoageing of the skin, said tocopheryl phosphate being at least partially encapsulated in lipid vesicles.

According to an essential characteristic of its second aspect, the invention relates to a cosmetic composition consisting of or comprising a continuous aqueous phase in which lipid vesicles are present, containing a tocopheryl phosphate, in particular in the form of a cosmetically acceptable salt or ester thereof as defined above, said tocopheryl phosphate being at least partially incorporated into these lipid vesicles and said composition also comprising at least one water-soluble polysaccharide.

Thus, the new cosmetic compositions which are the subject of the second aspect of the invention contain a tocopheryl phosphate, in particular an alpha-tocopheryl phosphate, at least partially included in lipid vesicles which are themselves contained in an aqueous phase.

This second aspect comprises two variants.

According to the first variant, the cosmetic composition consists of an aqueous phase in which the lipid vesicles are present.

According to the second variant of this second aspect, the cosmetic composition contains a continuous aqueous phase in which the lipid vesicles are present and which is more specifically in the form of an oil-in-water emulsion.

In the two variants of the new composition, the lipid vesicles, in particular when they are liposomes, are stabilised by the presence of an alginate in the aqueous phase.

Finally, according to an essential characteristic of the third aspect, the invention covers a cosmetic skincare process for preventing or slowing down the appearance of the effects of skin ageing, in particular of photoageing of the skin, comprising the application, to the part of the skin involved, of a cosmetic composition containing a tocopheryl phosphate as defined above.

According to the invention, for all of its aspects, said lipid vesicles may in particular be liposomes.

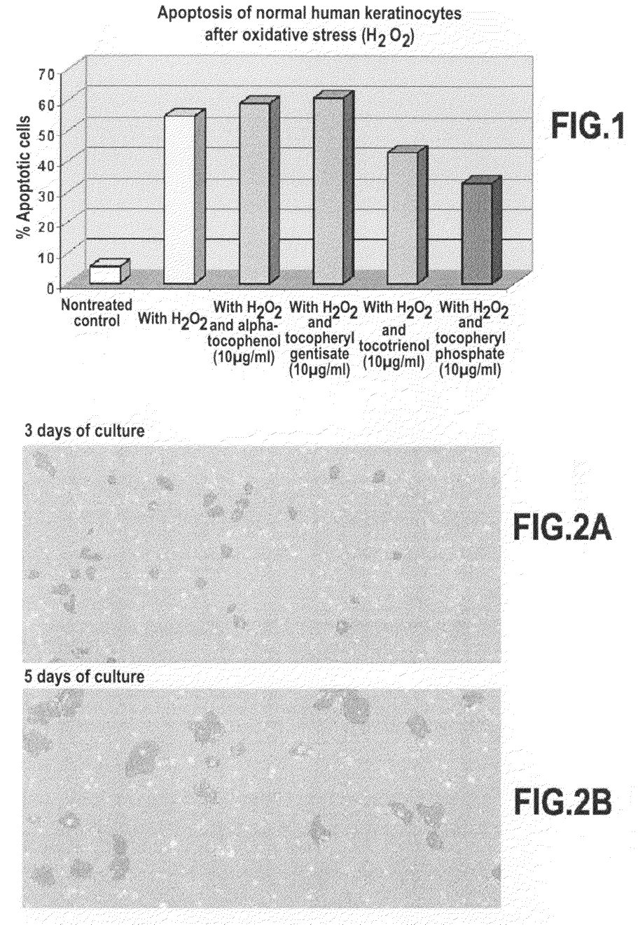

Other characteristics and advantages of the invention appear in the description and the examples which follow and also in FIGS. 1 to 8 to which reference is made in the examples.

FIG. 1, which refers to Example 1, illustrates the effectiveness of alpha-tocopheryl phosphate compared with other vitamin E derivatives as an agent for protecting epidermal stem cells;

FIG. 2 refers to Example 2 and represents the visualisation by videomicroscopy of the keratinocyte colonies derived from the epidermal stem cells at 3 days (FIG. 2A) and 5 days of culture (FIG. 2B);

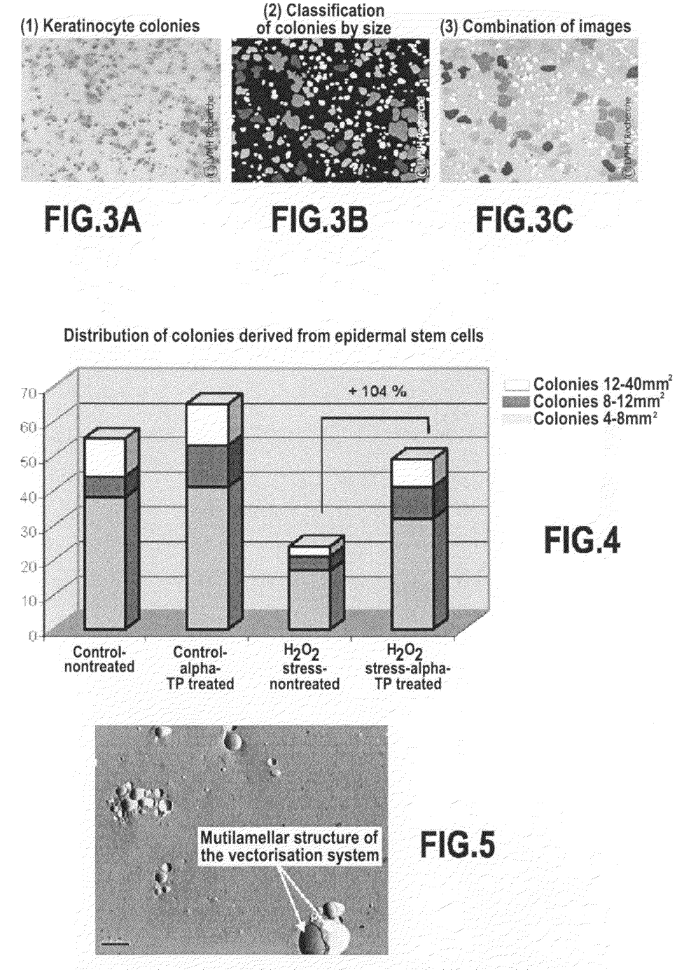

FIG. 3 (FIGS. 3A, 3B and 3C) gives the result of the visualisation of the keratinocyte colonies, FIG. 3A showing these colonies, FIG. 3B giving a classification of the colonies by size, and FIG. 3C, obtained after superimposition of the images, corresponding to FIGS. 3A and 3B;

FIG. 4 refers to Example 2 and gives the distribution of the colonies derived from epidermal stem cells after various treatments;

FIG. 5 is an image obtained by scanning electron microscopy after cryofracture using the serum prepared according to Example 3;

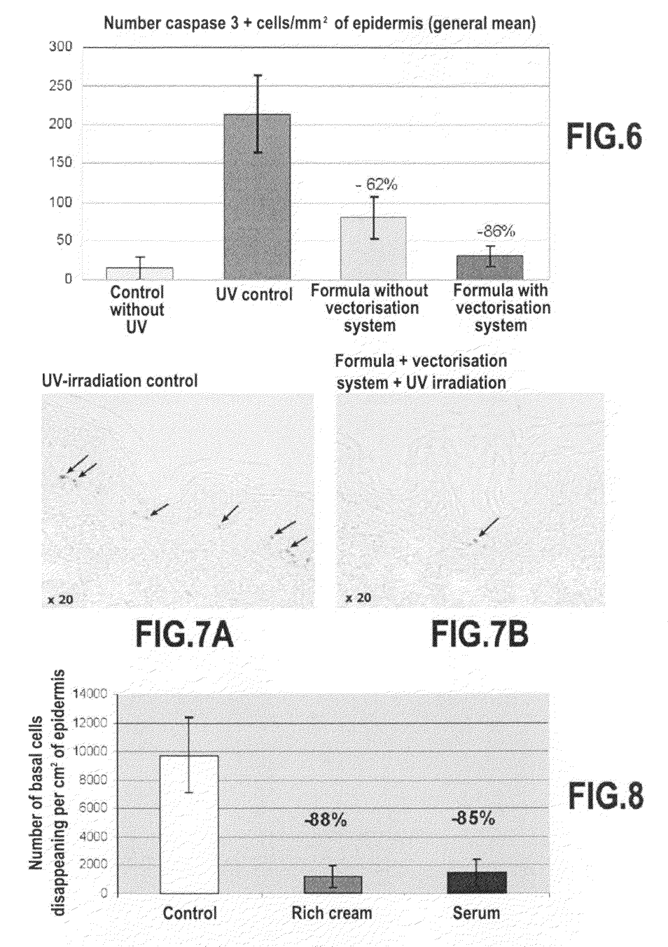

FIG. 6 is given with reference to Example 5 and gives the result of counting caspase 3+ cells after various treatments;

FIG. 7 gives the results of the immunolabelling of caspase 3+ (FIG. 7D) in comparison with a control (FIG. 7A);

FIG. 8 is given with reference to Example 6 and gives the number of basal cells disappearing per cm² of epidermis in the case of a control sample and in the case of two samples treated with alpha-tocopheryl phosphate.

In the various aspects of the invention, the tocopheryl phosphate will especially be in its dl form or in its d form or in the form of a cosmetically acceptable salt or ester thereof.

According to a particularly advantageous variant, the tocopheryl phosphate is alpha-tocopheryl phosphate.

The tocopheryl phosphate, in particular the alpha-tocopheryl phosphate, may be in the form of cosmetically acceptable salts chosen from the group consisting of alkali metal salts, in particular monosodium or disodium salts, alkaline earth metal salts, in particular magnesium salts, and ammonium salts or salts of primary, secondary or tertiary amines such as, in particular, diethylamine, diethanolamine, triethylamine or triethanolamine.

The uses covered by the first aspect of the present invention are all those in which it is sought to prevent or slow down the appearance of the effects of skin ageing, in particular of photoageing of the skin.

The invention results from the discovery, by its inventors, that tocopheryl phosphate, in particular alpha-tocopheryl phosphate, acts as a notable cosmetic agent for preventing or slowing down the appearance of the effects of skin ageing, in particular of photoageing of the skin.

The cosmetic compositions into which it is introduced make it possible in particular to prevent or slow down the formation of wrinkles on the face or to reduce or smooth out the wrinkles already formed.

As set out above, the uses covered in the first aspect of the invention are the uses of tocopheryl phosphate, in particular of alpha-tocopheryl phosphate, independently of the type of cosmetic composition, as an agent for combating skin ageing, whereas certain compositions, in particular those containing an aqueous phase containing lipid vesicles and at least one polysaccharide, are claimed as new products. For this reason, a distinction will subsequently be made between the compositions covered by the first aspect and hereinafter denoted “composition of the invention” and the compositions covered by the second aspect, which will be denoted “new compositions of the invention”.

Thus, the cosmetic compositions of which the use is covered by the present invention may be of all types.

However, according to an advantageous variant, the cosmetic composition will consist of or will comprise an aqueous phase in which lipid vesicles are present.

The lipid vesicles may be unilamellar or multilamellar, and are preferably multilamellar lipid vesicles.

The lipid vesicles used according to the invention have a diameter of approximately 150 to 200 μm, as measured by laser granulometry as a suspension of lipid vesicles or by Transmission Electron Microscopy with preparation of the sample by cryofracture.

According to the invention the lipids forming the lipid vesicles consist of amphiphilic lipids, i.e. molecules having a hydrophilic group which is indifferently ionic or non-ionic, and a lipophilic group.

In the present description and the claims, the term “lipid” covers all substances comprising a lipophilic group, the “fatty” carbonaceous chain of which contains more than 5 carbon atoms.

Similarly, for the purpose of the invention, the term “fatty alcohols” is intended to mean alcohols of which the carbonaceous chain contains at least 5 carbon atoms.

The amphiphilic lipids may be phospholipids, phosphoaminolipids, glycolipids, or mixtures of these lipids. Such substances consist, for example, of an egg or soybean lecithin, a sphingomyelin, a cerebroside or an oxyethylenated polyglyceryl stearate.

The phospholipids may be phosphatidylcholine, phosphatidylserine, phosphatidylethanolamine, phosphatidylglycerol, phosphatidylinositol, or mixtures thereof.

The bilayers of the lipid vesicles preferably consist of phospholipids originating from lecithin, in particular originating from soybean-lecithin.

In a preferred embodiment, these phospholipids are a mixture of two types of soybean lecithin, the first type of soybean lecithin being a mixture of phospholipids comprising more than 90% of phosphatidylcholine, the second type being a mixture of phospholipids comprising between 15% and 30% of phosphatidylcholine.

Lecithins in accordance with those used in the lipid vesicles of the invention are, for example, sold by the company Lucas Meyer under the trade marks Emulmetik® 300 and Emulmetik® 930.

According to an advantageous variant, the tocopheryl phosphate is at least partially encapsulated in these lipid vesicles.

According to the invention, in general, said lipid vesicles may in particular be liposomes.

It became apparent to the inventors of the present application, and this is the subject of a second patent application filed on the same day as the present application, that the presence of a water-soluble polysaccharide in the aqueous phase of an oil-in-water dispersion in which the aqueous phase contains liposomes in dispersion made it possible to very markedly increase the stability of these liposomes.

It has now emerged that, in general, the presence of at least one polysaccharide in water-soluble form in sufficient amount makes it possible to improve the stability of the lipid vesicles, and in particular of the liposomes, in the compositions of the invention.

The polysaccharide(s) in water-soluble form can be chosen from a wide range of water-soluble polysaccharides. They may in particular be chosen from the group consisting of starch or a derivative thereof, cellulose derivatives, pectins, gums, alginate, dextrans, carraghenates and hyaluronic acid.

Among the cellulose derivatives, carboxymethylcellulose, hydroxymethylcellulose, cellulose acetate or methylcellulose will in particular be chosen.

Among the gums, xanthan gum or guar gum will in particular be chosen.

Alginates, in particular alkali metal alginates, and most particularly a sodium or potassium salt or an extract containing same, for example an algal extract, will, however, advantageously be chosen.

In a preferred embodiment, the composition contains at least one alkali metal alginate and at least a second polysaccharide in water-soluble form, in particular an alkali metal salt of carboxymethylcellulose, preferably sodium carboxymethylcellulose.

The polysaccharide concentration will be chosen so as to effectively protect the lipid vesicles against degradation thereof.

Preferably, the total amount of water-soluble polysaccharide is between 0.1% and 10% by weight of the composition, preferably between 0.1% and 2% by weight.

Care will also be taken to ensure that the [phospholipids/water-soluble polysaccharide] ratio of the composition is between 0.1 and 20, preferably between 1 and 10.

The cosmetic composition according to the invention which necessarily comprises at least one water-soluble polysaccharide may also advantageously contain at least one hydrophilic polymer, preferably chosen from the group consisting of polyvinylpyrrolidone and polyvinyl alcohol, and mixtures thereof.

As disclosed above, the compositions of the invention are advantageously in the form of an emulsion comprising a continuous aqueous phase containing said lipid vesicles and said tocopheryl phosphate, and a dispersed fatty phase.

Among the compositions of the invention containing lipid vesicles, those in the form of an oil-in-water emulsion proved to be particularly advantageous in the applications targeted.

These aqueous compositions in emulsion form advantageously contain in their aqueous phase at least one non-ionic surfactant chosen from fatty alcohol polyethylene glycol ethers and fatty alcohol polypropylene glycol ethers, and mixtures thereof, and at least one polysaccharide in water-soluble form as defined above.

The non-ionic surfactant advantageously consists of a mixture of fatty alcohol polyalkylene glycol ethers advantageously having different HLBs.

The non-ionic surfactant is preferably chosen from fatty alcohol polyethylene glycol ethers and mixtures thereof, in particular the ethoxylated derivatives of the stearyl alcohol of formula (A) and mixtures thereof

CH₃(CH₂)₁₇(OCH₂CH₂)ₙOH (A).

In a particularly advantageous embodiment, a mixture of non-ionic surfactants is used, one being rather of substantially hydrophilic nature, and the second being rather of substantially lipophilic nature.

In a preferred embodiment of the invention, the non-ionic surfactant is a mixture of non-ionic surfactants comprising steareth-2, in accordance with formula (A) and in which n=2 on average, sold under the name Brij® 72 and the HLB value of which is 4.9, and steareth-21, in accordance with formula (A) and in which n=21 on average, sold under the name Brij® 721P and the HLB value of which is 15.3.

The steareth-2/steareth-21 ratio is adjusted so as to stabilise the emulsion without degrading the lipid vesicles.

In the compositions of the invention in emulsion form, the polysaccharide in soluble form as defined above is in sufficient amount to protect the lipid vesicles against degradation thereof under the effect of the surfactant.

The fatty phase of the emulsions of the invention advantageously contains triglycerides.

According to a variant of the invention, this fatty phase may itself be a water-in-oil (W/O) emulsion.

The cosmetic composition may also contain at least one hydrophilic polymer, in particular chosen from the group consisting of polyvinylpyrrolidone and polyvinyl alcohol, and mixtures thereof.

In addition, the cosmetic composition according to the invention may comprise other water-soluble hydrophilic compounds.

The soluble molecules may, for example, be a C₆ or C₁₂ sugar, advantageously chosen from glucose, sorbitol, sucrose, lactitol and glycerol or one of their ethers or esters or of their derivatives.

These water-soluble molecules are preferably obtained from a plant extract.

In addition to the tocopheryl phosphate, the cosmetic composition according to the invention may comprise one or more other active substances for preventing or slowing down the appearance of the effects of skin ageing, in particular of photoageing of the skin, and in particular for preventing or slowing down the formation of wrinkles on the face, or for reducing or smoothing out the wrinkles already formed.

As substance for reducing wrinkles, use may advantageously be made of the palmitoyl pentapeptide-3 sold under the name (Remixyl®) Matrixyl, in particular (Remixyl) Matrixyl® 3000, an extract of mallow (Vitactyl®), an extract of maize grain (Deliner®, Zea mays kernel extract) or an extract of oat bran (Osilift®).

Finally, the composition according to the invention may also comprise, in addition, one or more cosmetically acceptable excipients chosen from the group consisting of pigments, dyes, rheological agents, fragrances, sequestering agents, electrolytes, pH adjusters, antioxidants, preservatives, and mixtures thereof, texturing agents, and antisun agents or sunscreens.

In a preferred embodiment of the invention, the cosmetic composition is a lotion, a serum, an aqueous gel or else an oil-in-water (O/W) emulsion, preferably a serum or an oil-in-water emulsion.

As disclosed above, according to its second aspect, the invention relates to cosmetic compositions as new products.

These are, among the cosmetic compositions of the invention described above, those containing a tocopheryl phosphate, in particular in the form of a cosmetically acceptable salt or ester thereof as defined above, said tocopheryl phosphate being at least partially incorporated into lipid vesicles as defined above.

According to the invention, said lipid vesicles may in particular be liposomes.

In these compositions, the water-soluble polysaccharide is in an effective amount for stabilising said lipid vesicles.

Moreover, particularly advantageously, the water-soluble polysaccharide is present in the composition in an amount of between 0.1% and 10% by weight, preferably between 0.1% and 2% by weight.

Moreover, also particularly advantageously, the [phospholipids/water-soluble polysaccharide] ratio of the composition is between 0.1 and 20, preferably between 1 and 10.

The preferred compositions of the invention are in the form of an emulsion.

Finally, according to a last aspect, the invention covers a cosmetic skincare process for preventing or slowing down the appearance of the effects of skin ageing, in particular of photoageing of the skin, comprising the application, to the part of the skin involved, of a cosmetic composition of the invention.

### EXAMPLES

### Example 1

### Comparative Tests Concerning the Protection of Epidermal Stem Cells

The effectiveness of alpha-tocopheryl phosphate relative to other vitamin E derivatives as an agent for protecting epidermal stem cells, located in the basal layer, is compared.

Method

The cells undergoing apoptosis (programmed cell death) are examined on normal human keratinocytes (NHKS) in culture.

The cells are pretreated or not pretreated (controls) for 24 h with the vitamin E derivatives in solution at 10 μg/ml.

The cells are subsequently subjected to oxidative stress (1 mM H₂O₂ for 3 h).

The proportion of cells undergoing apoptosis is determined by cytometry using the fluorescent probes JC-1 (measurement of mitochondrial transmembrane potential) and TOTO-3 (measurement of plasma membrane permeability).

The apoptotic cells are JC-1-negative (corresponding to a drop in the mitochondrial transmembrane potential) and TOTO-3-negative (corresponding to an absence of alteration of the plasma membrane).

This method has been published (Zuliani et al. Cytometry part A, 2003, 54A: 100-106).

Results

The measurement results are given in the table below:

| | % NHKs undergoing |
|---|---|
| Experimental conditions | apoptosis |
| Nontreated control | 5.48 |
| Nontreated H₂O₂ control | 54.82 |
| With H₂O₂ and alpha-tocopherol | 58.82 |
| With H₂O₂ and tocopheryl gentisate | 60.67 |
| With H₂O₂ and tocotrienol | 42.85 |
| With H₂O₂ and tocopheryl phosphate | 32.79 |

A lower percentage of cells undergoing apoptosis is noted in the culture of cells treated with the vitamin E phosphate (see FIG. 1 giving the percentage of cells undergoing apoptosis after oxidative stress).

The vitamin E phosphate thus exerts a protective effect against oxidative stress for the keratinocytes, this effect being significantly greater than that observed for the control, and also than that observed for vitamin E or the other derivatives.

### Example 2

### Effect of Alpha-tocopheryl Phosphate on the Protection of Epidermal Stem Cells in Vitro

In order to study these epidermal stem cells in the basal cell layer of the epidermis, the test carried out, known as clonogenecity test, is based on the ability that the stem cells have of adhering, in vitro, to a culture support and of dividing so as to generate a large population of daughter cells grouped together in the form of colonies (Barrandon Y., Green H., Three clonal types of keratinocyte with different capacities for multiplication, J. Cell Biol. 84, 2302-2306 (1987), and Barrandon Y., Biologie des cellules souches epidermiques [Epidermal Stem Cell Biology], Ann. Dermatol Venereol. Suppl. 2: 285-286, (1998)).

Analysis of the number and of the size of the colonies after culture makes it possible to characterize the epidermal stem cells.

It also makes it possible to evaluate the protective action of an active agent when these cells are undergoing a stress.

The first step of isolating these cells consists in preparing a suspension of epidermal cells (Germain L et al., Improvement of human keratinocyte isolation and culture using thermolysin, Burns 19, 99-104 (1993)).

These cells are subsequently seeded onto feeder cells (3T3 fibroblasts), the mitotic activity of which is blocked with mitomycin.

This living support makes it possible to select the basal cells of the epidermis containing the stem cells and also makes it possible to ensure the growth of the cells with a high dividing capacity, which then form colonies of daughter cells (Barrandon, 1987).

The cells exhibiting the highest dividing capacity form large colonies (>4 mm²).

They correspond to the starting stem cell population.

FIG. 2 corresponds to the visualisation by video microscopy of the keratinocyte colonies derived from the epidermal stem cells at 3 days (FIG. 3A) and 5 days of culture (3B). The colouring of the colonies, herein red, shows the strong growth of certain colonies which correspond to the stem cells.

The protective effect of αTP was demonstrated according to this methodology.

First of all, the keratinocytes are treated with 1 μg/ml of αTP for 24 h, or not treated (controls), and then subjected to oxidative stress (hydrogen peroxide) for 15 minutes.

They are re-seeded at very low density for individualised growth.

After 7 days of culture, the cell colonies formed from each initial keratinocyte are coloured and counted by image analysis according to their size so as to evaluate the amount of stem cells of the initial culture.

FIG. 3 shows a visualisation of the keratinocyte colonies.

The cell colonies are classified by colour according to colony size (blue: greater than 12 mm²; red: from 8 to 12 mm²; orange: from 4 to 8 mm²; yellow: less than 4 mm²). FIG. 3-C represents the superimposition of the images represented in FIG. 3-A (original) and in FIG. 3-B (after colouring). It is thus verified that all the colonies have been taken into account.

The treatment with sodium alpha-tocopheryl phosphate under these conditions makes it possible to significantly maintain a high number of large colonies (>4 mm²), despite the oxidative stress, of +104% compared with the controls where this treatment with alpha-tocopheryl phosphate (alpha-TP) did not take place.

Reference will be made to the graph in FIG. 4 which gives the counts for the various colonies derived from epidermal stem cells as a function of their size: from 4 to 8 mm², from 8 to 12 mm² and greater than 12 mm².

Since the large colonies are derived from stem cells, these results thus indicate that the sodium alpha-tocopheryl phosphate therefore strongly protects this particular keratinocyte population.

### Example 3

### Preparation of a Serum Comprising Sodium Alpha-tocopheryl Phosphate

A serum is prepared according to the following formula (% expressed by weight relative to the total formula):

Phase A

| | |
|---|---|
| Purified water | 60.2% |
| Preservatives | 0.7% |

Phase B

| | |
|---|---|
| Carbomer (Carbopol ® Ultrez 10) | 0.5% |

Phase C

| | |
|---|---|
| Tetrasodium EDTA | 0.2% |
| Sodium hydroxide | 0.2% |
| Glycerol | 3.5% |
| Ascorbic acid | <0.1% |
| 1,3-Butylene glycol | 2.0% |
| Methyl gluceth-20 | 1.8% |
| Purified water | 6.0% |

Phase D

| | |
|---|---|
| Sodium tocopheryl phosphate | 0.2% |
| Sorbitol | 0.3% |
| Sodium alginate | 0.1% |
| Sodium carboxymethylcellulose | <0.1% |
| Polyvinyl alcohol | <0.1% |
| Emulmetik ® 300 IP | 0.5% |
| Emulmetik ® 930 | 0.5% |
| 1,3-Butylene glycol | 1.0% |
| Glycerol | 1.0% |
| Antioxidants | 0.2% |
| Purified water | qs 100% |

The phospholipids of phase D are homogenised with an Ultraturrax.

The phospholipids are subsequently homogenised with the butylene glycol and the glycerol for 20 minutes and the mixture is left to stand for at least 60 minutes.

The other compounds of phase D are added, followed by the purified water.

The lamellar phase obtained is sheared for 20 minutes with an Ultraturrax so as to form a dispersion of liposomes.

The sodium alpha-tocopheryl phosphate is included in the serum as an active agent and becomes encapsulated in the liposomes thus prepared.

Separately, the compounds of phase A are heated to 80° C.

At ambient temperature, the compounds of phase B are added, and then the gel is left to swell before adding the compounds of phase C to the gel previously formed.

The dispersion of liposomes is added to the aqueous phase previously prepared.

The composition thus obtained is a serum comprising multilamellar liposomes in which the sodium alpha-tocopheryl phosphate is encapsulated.

As appears in FIG. 5, in the present case, the liposomes comprise several phospholipid layers, which molecules are similar to those of cell membranes.

In this FIG. 5, which is a scanning electron microscopy image obtained on the serum prepared according to the present example, the presence of multilamellar spherical objects corresponding to the vectorisation system is noted.

### Example 4

### Rich Cream Comprising Liposomes Encapsulating Sodium Alpha-tocopheryl Phosphate

Phase A

| | |
|---|---|
| Steareth 2 flakes (Brij ®72 flakes) | 1.3% |
| Steareth 21 flakes (Brij ® 721P) | 2.2% |
| 95% Cetyl alcohol | 1.2% |
| Stearyl alcohol | 1.2% |
| Stearic acid | 0.35% |
| Palmitic acid | 0.35% |
| Cetyl palmitate | 1.3% |
| Hydrogenated polyisobutene | 5.3% |
| Dicaprylyl carbonate | 4.5% |
| Caprylic/capric triglycerides | 5.0% |
| Dimethicone | 0.2% |
| Cyclopentasiloxane | 2.1% |
| Preservatives | 0.7% |

Phase B

| | |
|---|---|
| Glycerol | 3.5% |
| Purified water | 40.6% |
| Acrylates/C10-C30 alkyl acrylate crosspolymer | 0.5% |

Phase C

| | |
|---|---|
| Tetrasodium EDTA | 0.2% |
| Sodium hydroxide | 0.1% |
| Caprylyl glycol | 0.5% |
| Purified water | 4.8% |

Phase D

| | |
|---|---|
| Sorbitol | 0.4% |
| Sodium alginate | 0.2% |
| Sodium carboxymethylcellulose | <0.1% |
| Polyvinyl alcohol | <0.1% |
| Emulmetik ® 300 IP | 0.5% |
| Emulmetik ® 930 | 0.5% |
| 1,3-Butylene glycol | 1.0% |
| Glycerol | 1.0% |
| Vitamin E phosphate, sodium salt | 0.2% |
| Antioxidants | 0.2% |
| Purified water | qs 100% |

The phospholipids of phase D are homogenised with an Ultraturrax.

The phospholipids are subsequently homogenised with the butylene glycol and the glycerol for 20 minutes and the mixture is left to stand for at least 60 minutes.

The other compounds of phase D are added, followed by the purified water.

The lamellar phase obtained is sheared for 20 minutes with an Ultraturrax so as to form a dispersion of liposomes.

Phases A and B are heated to 85° C. separately so as to obtain two homogeneous solutions.

Phase B is subsequently emulsified in oily phase A.

The O/W emulsion obtained is gradually cooled with stirring, and then, at 70° C., the compounds of phase C are added, in particular to neutralise the polymers.

The dispersion of liposomes is added to the O/W emulsion previously prepared, with stirring and without incorporating air.

The emulsion obtained comprises multilayer liposomes in the continuous aqueous phase.

These liposomes are not destroyed by the action of the surfactants stabilising the emulsion.

### Example 5

### Effect of Alpha-tocopheryl Phosphate (αTP) Encapsulated in Liposomes on the Protection of Epidermal Basal Cells on a Skin Explant, after UV Attack

The advantage of the encapsulation of the sodium alpha-tocopheryl phosphate in liposomes is compared here.

The serum comprising the sodium alpha-tocopheryl phosphate is prepared in accordance with Example 3.

For the test, a serum devoid of liposomes (phase D of the previous example), in which the sodium alpha-tocopheryl phosphate is solubilised in phase B, is prepared.

Method

Tissue from plastic surgery on a 36-year-old woman (Caucasian, P495AB36) was cut up into 15 explants 10 mm in diameter. These explants are kept alive in BEM medium (BIO-EC's Explants Medium).

1. Treatment

At time 0, the explants are placed in the survival medium (2 ml/explant) and the treatment is applied:
The explants are treated twice a day, by topical application (2 mg/explant) for 2 days before irradiation.
On the day of irradiation, one application is carried out 3 h before the irradiation and one application just after irradiation.
The following day, an application is carried out 3 h before the end of the survival period.
The controls do not receive any treatment.

2. Irradiation

The irradiation system is an Oriel solar simulator, normally used to determine the sun protection factor (SPF) and for photosensitisation studies.

At day 2, before irradiation, the survival medium is replaced with HBSS buffer (Hank's Balanced Saline Solution). The irradiation dose is 7 J/cm².

Just after the irradiation, the explants are again placed in the survival medium. The nonirradiated explants are placed in the dark during the irradiation and their medium is also replaced with HBSS buffer.

3. Samples

At day 3, 24 h after the end of irradiation, the explants are removed and fixed in formol buffer.

4. Immunolabelling of active caspase-3

The explants are dehydrated in a Leica automat TP 1020, embedded in paraffin (Leica EG 1160 embedding station automat).

Sections 5 μm thick are cut on a microtome (Leica Minot type microtome RM 2125) and placed on silanised slides.

The caspase-3 immunolabelling of the cells is carried out on the sections using a polyclonal anti-active caspase-3 antibody (rabbit, Chemicon ref AB3623) which recognises the active form of caspase-3.

The labelling is revealed using a Vectastain universal ABC VECTOR amplifier system kit with DAB staining.

The images are taken on a Nikon TE2000 videomicroscope under transmitted light for the immunolabelling combined with a phase-contrast image for measuring the surface of the epidermis.

The caspase-3-positive cells are counted by image analysis on the LEICA QWIN software and quantified relative to the surface of the epidermis.

Results

The caspase-3+ cells are counted on the samples of cells treated with the formulations tested.

The results are represented in FIG. 6, which gives the number of epidermal basal cells disappearing in fragments of skin exposed to a complete solar spectrum. These cells are identified by immunohistochemistry using an antibody which recognises active caspase-3, a marker for apoptotic cells.

FIG. 7 gives the result of the immunolabelling of active caspase-3 on skin explants exposed to a complete solar spectrum. Only the basal cells of the epidermis are labelled. A smaller number of positive cells is noted in the epidermis treated with the vectorisation system.

We demonstrated that a composition containing the vectorisation system makes it possible, when it is applied to human skin samples kept alive ex vivo and exposed to a complete solar spectrum, to significantly increase the protective efficacy of sodium alpha-tocopheryl phosphate with respect to the epidermal stem cells located in the basal layer (FIGS. 6 and 7).

### Example 6

### Effect of Alpha-tocopheryl Phosphate (αTP=TP Vityl) Encapsulated in Liposomes on the Protection of Epidermal Basal Cells on a Skin Explant, after UV Attack

The protective effect, with respect to epidermal stem cells, of cosmetic compositions comprising the alpha-tocopheryl phosphate encapsulated in liposomes and prepared according to Examples 3 and 4 of the present patent is evaluated.

These compositions are a serum and an oil-in-water emulsion in which the continuous aqueous phase comprises said liposomes.

The same test as in Example 5 is carried out on these compositions.

The results are reproduced in FIG. 8, which represents the number of basal keratinocytes of the epidermis which disappear during acute exposure with a complete solar spectrum after treatment with the serum or the rich cream comprising the tocopheryl phosphate encapsulated in liposomes. These cells are identified by indirect immunofluorescence using anti-active caspase-3 antibody, active caspase-3 being a marker for apoptotic cells.

We demonstrated that the rich cream and the serum containing 0.2% of αTP, applied to fragments of skin, reduce by more than 80% the number of basal cells of the epidermis which disappear after acute exposure with a complete solar spectrum.

1. A method of preventing or slowing down the effects of skin ageing, comprising applying to the skin a cosmetic composition, comprising a tocopheryl phosphate, or a cosmetically acceptable salt or ester thereof, as a cosmetic agent for preventing or slowing down the appearance of the effects of skin ageing, said tocopheryl phosphate being at least partially encapsulated in lipid vesicles.

2. The method according to claim 1, which is for preventing or slowing down the formation of wrinkles on the face, or for reducing or smoothing out the wrinkles already formed.

3. The method according to claim 1, wherein said tocopheryl phosphate is alpha-tocopheryl phosphate.

4. The method according to claim 1, wherein said tocopheryl phosphate is in a form chosen from the group consisting of the alkali metal salts, the alkaline earth metal salts, and the ammonium salts or salts of primary, secondary or tertiary amines.

5. The method according to claim 1, wherein said lipid vesicles are liposomes.

6. The method according to claim 1, wherein said cosmetic composition comprises a continuous aqueous phase in which said lipid vesicles are present.

7. The method according to claim 1, wherein said lipid vesicles are multilamellar lipid vesicles.

8. The method according to claim 1, wherein the lipid vesicles are formed from amphiphilic lipids chosen from the group consisting of phospholipids, phosphoaminolipids, glycolipids, and mixtures thereof.

9. The method according to claim 1, wherein the lipid vesicles are formed from amphiphilic lipids chosen from the group consisting of egg lecithin, soybean lecithin, sphingomyelin, cerebrosides and oxyethylenated polyglyceryl stearates.

10. The method according to claim 6, wherein said cosmetic composition also contains, in said aqueous phase, at least one polysaccharide in water-soluble form, in an effective amount for improving the stability of said lipid vesicles.

11. The method according to claim 10, wherein said polysaccharide is chosen from the group consisting of starch or a derivative thereof, cellulose derivatives, pectins, gums, alginate, dextrans, carraghenates and hyaluronic acid.

12. The method according to claim 10, wherein said composition contains at least one alginate in water-soluble form.

13. The method according to claim 12, wherein the alkali metal alginate is sodium alginate.

14. The method according to claim 10, wherein said composition comprises a second polysaccharide in water-soluble form.

15. The method according to claim 1, wherein said cosmetic composition is in the form of an emulsion comprising a continuous aqueous phase containing said lipid vesicles and said tocopheryl phosphate, and a dispersed fatty phase.

16. The method according to claim 15, wherein said aqueous phase contains at least one non-ionic surfactant chosen from fatty alcohol polyethylene glycol ethers and fatty alcohol polypropylene glycol ethers, and mixtures thereof, and at least one polysaccharide in water-soluble form as chosen from the group consisting of starch or a derivative thereof, cellulose derivatives, pectins, gums, alginate, dextrans, carraghenates and hyaluronic acid.

17. The method according to claim 15, wherein said aqueous phase contains a mixture of non-ionic surfactants as chosen from the group consisting of starch or a derivative thereof, cellulose derivatives, pectins, gums, alginate, dextrans, carraghenates and hyaluronic acid.

18. The method according to claim 15, wherein said non-ionic surfactant is chosen from fatty alcohol polyethylene glycol ethers and mixtures thereof.

19. The method according to claim 15, wherein the non-ionic surfactant is chosen from stearyl alcohol polyethylene glycol ethers.

20. The method according to claim 19, wherein said non-ionic surfactant is a mixture of steareth-2 and steareth-21.

21. The method according to claim 15, wherein the composition comprises a continuous aqueous phase that contains at least one polysaccharide in water-soluble form, and the polysaccharide is chosen from the group consisting of starch or a derivative thereof, cellulose derivatives, pectins, gums, alginate, dextrans, carraghenates and hyaluronic acid and is in an effective amount for protecting the lipid vesicles against degradation thereof under the effect of said surfactant.

22. The method according to claim 15, wherein said cosmetic composition also contains at least one hydrophilic polymer.

23. The method according to claim 15, wherein the fatty phase of said emulsion contains triglycerides.

24. The method according to claim 15, wherein the fatty phase of said emulsion is itself a water-in-oil (W/O) emulsion.

25. Cosmetic composition containing a tocopheryl phosphate, or a cosmetically acceptable salt or ester thereof as a cosmetic agent for preventing or slowing down the appearance of the effects of skin ageing, said tocopheryl phosphate being at least partially incorporated into lipid vesicles, and said composition also comprising at least one water-soluble polysaccharide as defined as chosen from the group consisting of starch or a derivative thereof, cellulose derivatives, pectins, gums, alginate, dextrans, carraghenates and hyaluronic acid.

26. Cosmetic composition according to claim 25, characterized wherein said lipid vesicles are liposomes.

27. Cosmetic composition according to claim 25, wherein said water-soluble polysaccharide is in an effective amount for stabilising said lipid vesicles.

28. Cosmetic composition according to claim 25, wherein said water-soluble polysaccharide is present in the composition in an amount of between 0.1% and 10% by weight.

29. Cosmetic composition according to claim 25, wherein the [phospholipids/water-soluble polysaccharide] ratio of the composition is between 0.1 and 20.

30. Composition according to claim 25, wherein it is in the form of an emulsion comprising a continuous aqueous phase containing said lipid vesicles and said tocopheryl phosphate.

31. A method for preventing or slowing down the appearance of the effects of photoageing of the skin, comprising the application, to the part of the skin involved, of a cosmetic composition comprising a tocopheryl phosphate or a cosmetically acceptable salt or ester thereof, as a cosmetic agent for preventing or slowing down the appearance of the effects of photoageing of the skin, said tocopheryl phosphate being at least partially encapsulated in lipid vesicles.

32. The method according to claim 1, wherein the tocopheryl phosphate is in its dl or d form.

33. Composition according to claim 25, wherein the tocopheryl phosphate is in its dl or d form.

34. The method according to claim 31, wherein the tocopheryl phosphate is in its dl or d form.
